(19) United States
(12) Patent Application Publication
(10) Pub. No.: US 2008/0202967 A1
(43) Pub. Date: Aug. 28, 2008

(54) Cosmetic Product

(76) Inventor: Kerryne Krause-Neufeldt, Midrand (ZA)

Correspondence Address: NIXON PEABODY, LLP, 401 9TH STREET, NW, SUITE 900, WASHINGTON, DC, 20004-2128, US

(21) Appl. No.: 11/816,980

(22) PCT Filed: Feb. 22, 2006
(86) PCT No.: PCT/IB06/00354

§ 371 Date: Oct. 19, 2007

(30) Foreign Application Priority Data
Feb. 23, 2005 (ZA) 2005/01589
May 17, 2005 (ZA) 2005-03965

(51) Int. Cl. A61J 1/00; A61K 9/00; A61K 36/886; A61K 36/82; A61K 31/16; A61K 38/16; A61K 31/49; A61K 36/896; A61K 36/74; A61K 36/42; A61K 33/40; A61K 31/592; A61K 31/355; A61K 31/07; A61K 31/375

(52) U.S. Cl. 206/438; 424/400; 424/744; 424/729; 514/625; 514/12; 514/314; 424/758; 424/613; 514/167; 514/458; 514/725; 514/474; 424/725

(57) ABSTRACT

This invention relates to a treatment pad which may be used in cosmetic treatment and which produces soothing, relaxing, calming and similar effects in persons. The treatment pad comprises 0.1 to 90% weight (% wt) of a hydrophilic polymer selected from the group consisting of polyvinyl alcohol (PVA), polyethylene glycol (EG), polyacrylamide (PAAm), polyacrylic acid (PAAc), polyhydroxyethyl methacrylate (PHEMA) and polyethylene oxide (PEO); 0.01 to 0.99% wt preservative and the balance being water

### FIELD OF INVENTION

This invention lies in the field of a treatment pad which may be used in cosmetic treatment. In particular this invention relates to a treatment pad for producing soothing, relaxing, calming and similar effects in persons. In particular the invention relates to a treatment pad for application to the exterior regions of the eye and surrounding tissues. The invention may be applicable to other areas of the body.

### BACKGROUND OF THE INVENTION

Disposable eye masks are available, which consist of a textile or paper base impregnated with moisture and ingredients that sooth the eye area once applied. They may contain ingredients such as distilled rose water, cucumber extract, aloe extract or green tea. Their main disadvantages are that they dry out quickly and have to be used in a recumbent position. Plastic eye masks are known, which merely cover the eye region, without any effects beyond cooling, they are fastened by a string and lack comfort. Hydrogels have been used but cannot be re-used and tend to break up rather easily. A hydrogel patch has been proposed, which consists of a water-based gelatinous precipitate on a non-woven cloth, a protective sheet covers the gel and is peeled off before use. A hydrogel can have beneficial effects on the delicate skin around the eyes and on the eyelids. Certain creams and ointments are known, which have limited effects.

### SUMMARY OF THE INVENTION

According to a first aspect to the present invention there is provided a treatment pad comprising:
0.1 to 90% weight (% wt) of a hydrophilic polymer selected from the group consisting of polyvinyl alcohol (PVA), polyethylene glycol (EG), polyacrylamide (PAAm), polyacrylic acid (PAAc), polyhydroxyethyl methacrylate (PHEMA) and polyethylene oxide (PEO);
0.01 to 0.99% wt preservative; and
the balance being water.

Preferably the treatment pad is a pad adapted to treat the eye and/or regions of tissue surrounding the eye. The pad is preferably self supporting and may be ergonomically shaped to be accommodated within an eye well of a user thereof.

In a preferred embodiment the pad is adapted to adhere to skin, preferably human skin, for at least 30 seconds in the vertical or substantially vertical position, more preferably at least 1 minute, more preferably at least 2 minutes, most preferably 5 minutes. The pad may be adapted to exhibit this adherence through possessing sufficient tackiness (wet tack). Sufficient tackiness may be imparted to the pad through judicious choice of hydrophilic polymer and/or the concentration in which the polymer is present and/or the addition of a tackifying agent to the formulation.

The hydrophilic polymer is preferably present in an amount greater than 1% wt, preferably greater than 3% wt, more preferably greater than 5% wt. The hydrophilic polymer is preferably present in an amount less than 60% wt, more preferably present in an amount less than 50% wt, most preferably in an amount less than 40% wt. In a preferred embodiment of the present invention, the polymer is present in an amount of approximately 10% wt.

The polymer is preferably polyvinyl alcohol (PVA) and may be supplied by Air Products, for example Airvol (trade mark).

The preservative may be selected from those effective against gram negative and gram positive bacteria, yeasts and/or moulds or other unwanted life forms which may occur in or on the pad. The preservative is preferably suitable for cosmetic or topical application to the human body, in particular the skin.

As such, preservatives include, but are not limited to, parabenzoates such as methyl, ethyl, butyl and propyl parabenzoates, glycols, such as propylene glycol and ureas such as diazolidinyl urea.

In a preferred embodiment, the formulation includes a plurality or mixture of preservatives and may include commercially available preservative formulations such as Germaben and/or Kemaben (both trade marks) which includes methyl parabenzoate, propyl parabenzoates, propylene glycol and diazolidinyl urea.

Too higher concentration of preservative present in the formulation according to the present invention can be irritating to human tissue, in particular the skin, and particularly tissue and/or skin surrounding the eye. As such it is necessary that the treatment pad comprises less than 1.0% wt of preservative, preferably less than 0.9% wt, more preferably less than 0.8% wt preservative. In a preferred embodiment, the formulation contains approximately 0.5% wt preservative.

In this specification, reference to the amount of preservative present refers to the total amount of preservative present, in other words, if more than one preservative is present, the combined amount should not be greater than 0.99% wt.

The pad preferably includes further ingredients for ameliorating disorders or unsightly marking of tissue and/or skin, for example, surrounding the eye. These further ingredients may be directed toward improving micro circulation, benefiting collagen and elastin, free radical conditions and/or cellular metabolism in the tissue and/or skin. Such disorders or unsightly marking includes dark under-eye rings or circles and puffiness of the eyes. In terms of this invention, the term ‘puffiness’ should be read to include swelling and/or redness of the tissue surrounding the eye.

The pad may also be used to ameliorate symptoms of fatigue, such as scratchy and/or bloodshot eyes. In addition the treatment pad can be used in the treatment of Graves Disease where the pad was found to enormously reduce puffiness and swelling of the eyes associated with the disease.

Free radicals are single oxygen atoms that attach themselves the walls of cells and may change the cell structure causing damage to the cells. Free radicals are known to cause wrinkles, cancer and other damage and are caused by oxygen splitting into two single atoms which then attach themselves to the cells. Various vitamins and oxygen can counteract the effects of free radicals, destroying them and prohibiting them from doing damage. Such as vitamins include vitamins C, D, A & E.

Optimising cellular metabolism refers to the optimal functioning of the cell. In order for a cell to function at its best, it needs water, oxygen, vitamins and minerals and other nutrients. When a cell's metabolism is under par, certain conditions develop such as bad circulation, leading to dark circles under the eyes and cellulite.

The further ingredients which may be included in the treatment pad include derivatives from the Aloe plant including Aloe Vera, Aloe Arborescence and Aloe Ferox. Such Aloe derivatives include Aloesin which is a low molecular weight ingredient present in, inter alia, Aloe Vera.

Other ingredients include provitamins, for example, panthenol (a provitamin is herein defined as a compound which could become a vitamin if it were to enter a living cell), lipid dispersions, preferably derived from botanical sources, for example, Fitobroside (trade mark), proteins, preferably botanically derived proteins, for example, rice bran protein and soy protein, quinine and cinchona derivatives, suitable emollients, a source of collagen, preferably soluble collagen, for example Collasol (trade mark), vitamins, for example, vitamin A and/or vitamin C, green tea, cucumber extract, bulbine extract, stabilized peroxides, preferably encapsulated into an emulsion, which peroxides safely release oxygen into the skin and radical waters. Radical water is a technology based on the process of electrochemical activation of water in a patented reactor system that kills bacteria, spores, fungi, yeast and moulds. The pad may include a plurality or mixture of these ingredients and may include a commercially available formulation comprising any one or more of these ingredients, for example, Regu-Age (trade mark).

The pad may also contain suitable dyes, perfumes and other adjuvants known in the art.

The additional ingredients, dyes, perfumes and adjuvants may be present in an amount of from 0.01 to 20% wt each. In particular the additional ingredients, dyes, perfumes and adjuvants are preferably each present in an amount exceeding 0.1% wt, more preferably exceeding 0.3% wt, most preferably exceeding 0.5% wt. The additional ingredients, dyes, perfumes and adjuvants are preferably present in an amount less than 20% wt, more preferably less than 10% wt, more preferably less than 8% wt, most preferably less than 5% wt.

In a preferred embodiment of the invention, the pad includes specially purified soy and/or rice peptides and/or yeast protein, preferably biotechnologically produced yeast protein, in particular hydrolyzed rice bran protein, glycine soja protein, oxido reductases and a broad spectrum preservative, for example a phenoxyethanol, methyl-, butyl-, ethyl- or propyl parabenzoate.

The pad may also contain plant material and extracts from locally grown indigenous plants in South Africa, for example various fynbos and rooibos varieties. These include but are not limited to Buchu (Agathosma Betulina), Cape May (Coleonema Album), Zinziba (Lippia Javanica), Cape Chamomile (Eriocephalus Punctulatus), Cape geranium (Pelargoneum Graveolens) and Cape Snowbush (Eriocephalus Africanus).

The treatment pad can be used for at least an hour in total before it desiccates to the point of diminished effectiveness. It is intended that a user of the pad would use the pad for approximately 5 minutes before replacing it in an environment where desiccation is minimised. Under such conditions, each pad may be used for at least 12 sessions of 5 minutes each. However, it will be appreciated that the pad may be used for longer periods, for example 15 minutes, half an hour or even a continuous hour. In situations such as post-operative surgery healing, it is preferable to use the pad for much longer times and the ultimate life span of the pad is entirely dependent on conditions of use, for example, relative humidity. It is preferable that the pad is used at ambient conditions of not more than 23 degrees Celcius.

According a second aspect of the present invention there is provided a product for treatment of the human body comprising a treatment pad as hereinbefore described and a packaging unit which comprises at least one container with a lid. Preferably the product is a cosmetic product. Preferably the packaging unit is adapted to receive at least two treatment pads, one for each eye, preferably one in each container. Preferably the lid of the packaging unit engages with the container to substantially seal the contents of the container from the ambient, when the lid is closed. Preferably the packaging unit comprises oval containers with lids.

In accordance with a preferred embodiment of the invention, the packaging unit comprises two conjoined containers, each with a base and a hinged lid, the containers being conjoined by a hinge that allows the containers to be hinged from an open position with respect to each other with their lids facing upwards to a position in which they are contiguous with each other, back to back. The expression “back to back” refers to the outer surfaces of the bases touching or close to each other. A clip may be provided to normally keep them in this back to back position and allow them to be opened up to the open position with a light pressure. Alternatively, the containers may be retained in position by frictional engagement.

The treatment pad may either be directly received in the container or the container may receive a package containing the treatment pad. Such package may be manufactured using a form, fill and seal process, for example.

The present invention further provides a method of preparation of a treatment pad as hereinbefore described which comprises the steps of mixing the hydrophilic polymer, preservative and water and subjecting the mixture to at least one freeze thaw cycle.

Preferably the water and hydrophilic polymer are added in a heating vessel and heated to between 60 and 100 degrees Celcius (deg C.), preferably between 70 and 95 deg C., preferably between 80 and 90 deg C., most preferably approximately 85 deg C. for between 1 and 14 hours. In one embodiment of this aspect to the present invention the heating is conducted for between 9 and 13 hours, most preferably 12 hours. However, by using a shear mixer and a jacketed reactor mixture, preferably under vacuum, the heating time can be reduced to approximately 1 hour.

After cooling to approximately 40 deg C., the remaining ingredients may be added and mixed in. A suitable aliquot of the mixture is thereafter added to a container, for example, the containers of the packaging units, which units are preferably themselves included in a tray held open and flat. A suitable aliquot will vary according to needs but may be approximately 6 milliliters per container.

In an alternative embodiment to the invention, the mixture may be added to a form, fill and seal apparatus known in the art. Such an apparatus may thereafter produce discrete thermoplastic units filled with the correct aliquot and sealed against the ambient, either under an inert gas or vacuum. These formed, filled and sealed packages are preferably so dimensioned so as to be receivable in the containers of the packaging units.

It is contemplated in the ambit of the present invention that the formed, filled and sealed packages could be sold independently as refill units.

The containers containing the mixture or formed, filled and sealed packages may then be closed and subject to the predetermined number of freeze thaw cycles. By freeze thaw cycles it is intended that the mixture is subject to cold conditions, preferably not exceeding −30 deg C., more preferably not exceeding −20 deg C. Following at least one hour, preferably two hours, at these conditions the mixture may be allowed to thaw, for example for 1 hour. This constitutes one freeze thaw cycle. Preferably the mixture is not subject to more than three freeze thaw cycles. Following the last thaw, the containers may be clipped closed and sealed for shipping.

The product freezes at −10 deg C. in an hour and freezes at −20 deg C. within half an hour. It may also be possible to employ blast freezing at lower temperatures (up to −30 deg C. or more) to speed up the process. Heat may be used in the thawing process to speed up the thaw times and the use of such heat does not negatively impact on the end product. Thaw time can be as fast as 10 minutes and as slowly as 1 hour. Freeze times can be as fast as 10 minutes and as slowly as 2 hours at ranges between −10 deg C. to −30 deg C.

Either following or before the freeze thaw cycles the pad may be dispensed into the packaging units. The packaging units may be held in trays. Preferably the trays are adapted to facilitate the freeze thaw processes.

According to a further aspect to the present invention there is provided an apparatus comprising a tray adapted to receive a plurality of packaging units as hereinbefore described for use in the method of the invention. The packaging units are preferably held firmly in place, flat and open. It is an advantage of the invention to use the conjoined containers of the packaging units which containers can be filled in the open position and after the processing of the pad is complete, folded closed and be ready for dispatch to consumers.

The apparatus is preferably dimensioned to be easily accommodated within a refrigerator suitable for use in the freeze thaw processes.

### BRIEF DESCRIPTION OF THE DRAWINGS

The invention is more fully described by way of non-limiting examples, with reference to the drawings, in which:

FIGS. 1(a) and (b) are plan and elevation views respectively of a first embodiment of a packaging unit,

FIG. 2 is an isometric view of the packaging unit, with one pad lifted up for illustrative purposes,

FIG. 3 is an isometric view of the packaging unit partially folded on its hinges,

FIGS. 4(a) and (b) are plan and side views of the packaging unit fully closed and folded back to back,

FIG. 5 is a diagrammatic plan of a refrigeration unit for use in the present invention,

FIG. 6 is a diagrammatic plan view of a tray including a plurality of packaging units,

FIG. 7 is a diagrammatic plan view of a tray without the packaging units,

FIG. 8 is a diagrammatic plan view of a trolley adapted to receive a plurality of trays,

FIGS. 9(a) and (b) are diagrammatic elevation and side elevation views of a trolley assembly, respectively,

FIG. 10 is a diagrammatic elevation of the trolley received within a refrigeration unit,

FIGS. 11(a-d) are different views of an alternative embodiment of a packaging unit,

FIGS. 12(a) and (b) are side and front views respectively of an alternative embodiment of a trolley assembly and

FIGS. 13(a) and (b) are front and plan diagrammatic views of freezer units including several trolley assemblies as shown in FIG. 12.

### DESCRIPTION OF PREFERRED EMBODIMENTS

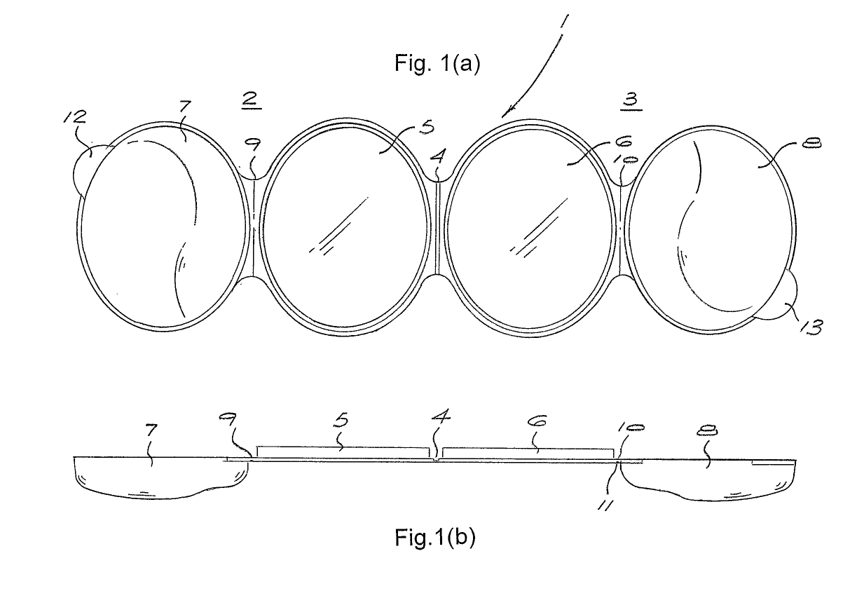

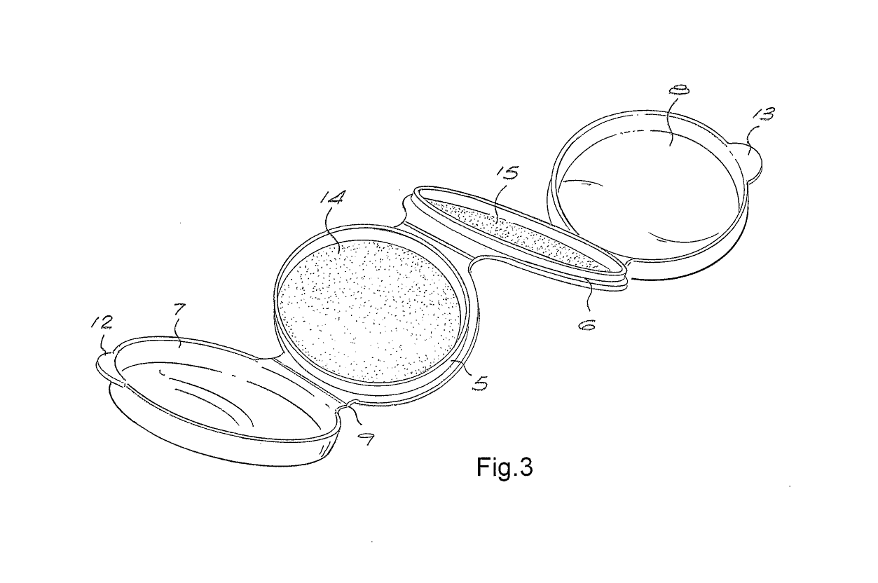

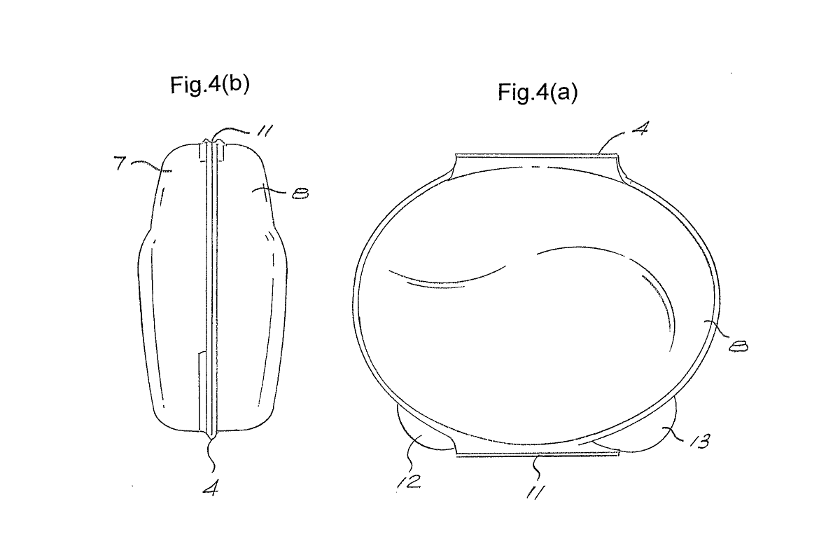

As shown in FIGS. 1 to 4, the packaging unit 1 consists of two conjoined containers 2 and 3, which provide for two hydrophilic polymer pads, one for each eye of a user. The two containers are conjoined by a plastic hinge 4. Each container consists of a base 5, 6 and a hinged lid 7, 8. The lids are hinged to the bases by plastic hinges 9, 10. The lids seal onto the bases when closed onto the bases. The containers being conjoined by the hinge allows the containers to be hinged from an open position as shown in FIGS. 1(a) and 1(b) with respect to each other with their lids facing upwards to a position in which they are contiguous with each other, back to back as shown in FIGS. 4(a) and 4(a). As can be seen in FIG. 4(b) the expression “back to back” refers to the outer surfaces of the bases 5, 6 touching each other. A clip 11 is provided to normally keep them in this position and allow them to be opened up to the open position. Each lid also has a tab 12, 13 to aid in opening the lid with a light pressure. FIG. 3 shows the containers beginning to be folded into the back to back position.

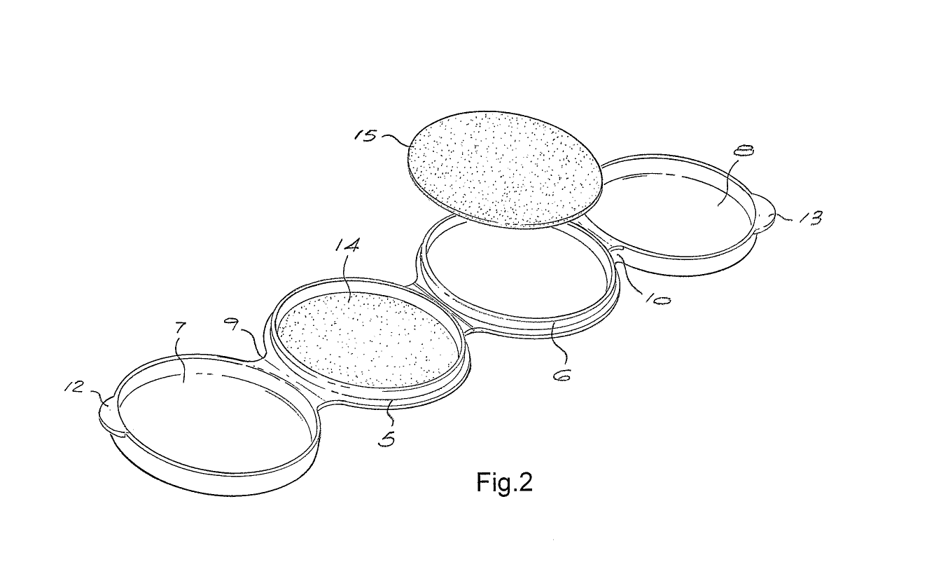

The packaging units are held in the position shown in FIGS. 1(a) and 1(b) for filling with the hydrophilic polymer during the process of manufacturing the product and FIG. 2 shows the hydrophilic polymer pads 14, 15, the latter shown raised. When the product is to be used it is lifted out of the containers and applied to the eyes.

An example of a liquid antimicrobial preservative that is used in the polymer is “Kemaben”, (trade mark), which is a blend of 30% diazolidinyl urea, 11% methyl paraben, 3% propylparaben and 56% propylene glycol. An example of a product to add wet tack, open time, heat resistance and emulsion compatibility is “Airvol” (trade mark). An example of an agent to reduce puffiness, dark rings and other skin benefits is “Regu-age” (trade mark). An example of a broad spectrum preservative is “Phenonip” (trade mark). These additives have a cosmetic effect for the user as well as soothing and similar effects.

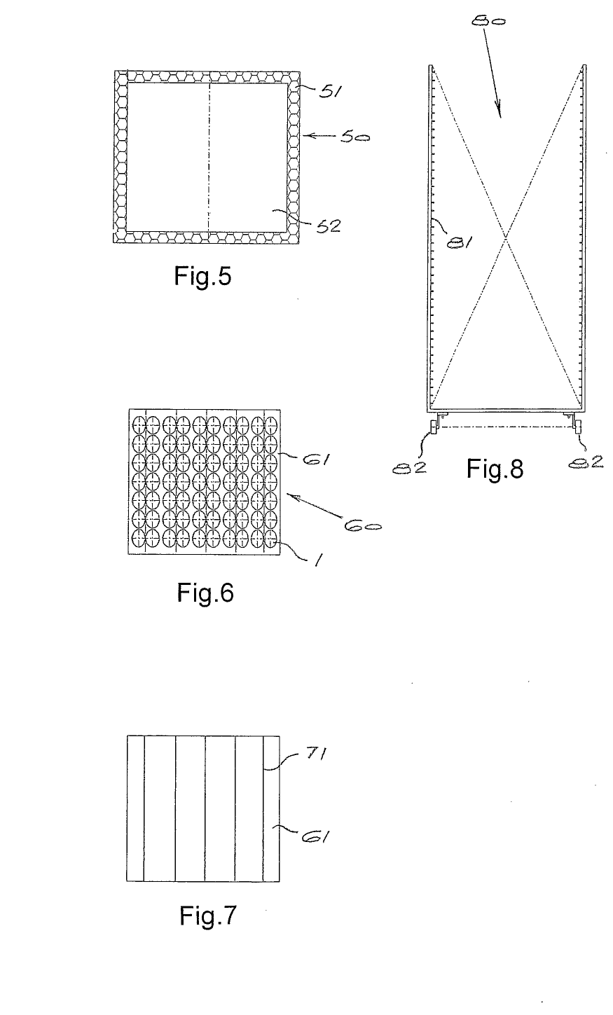

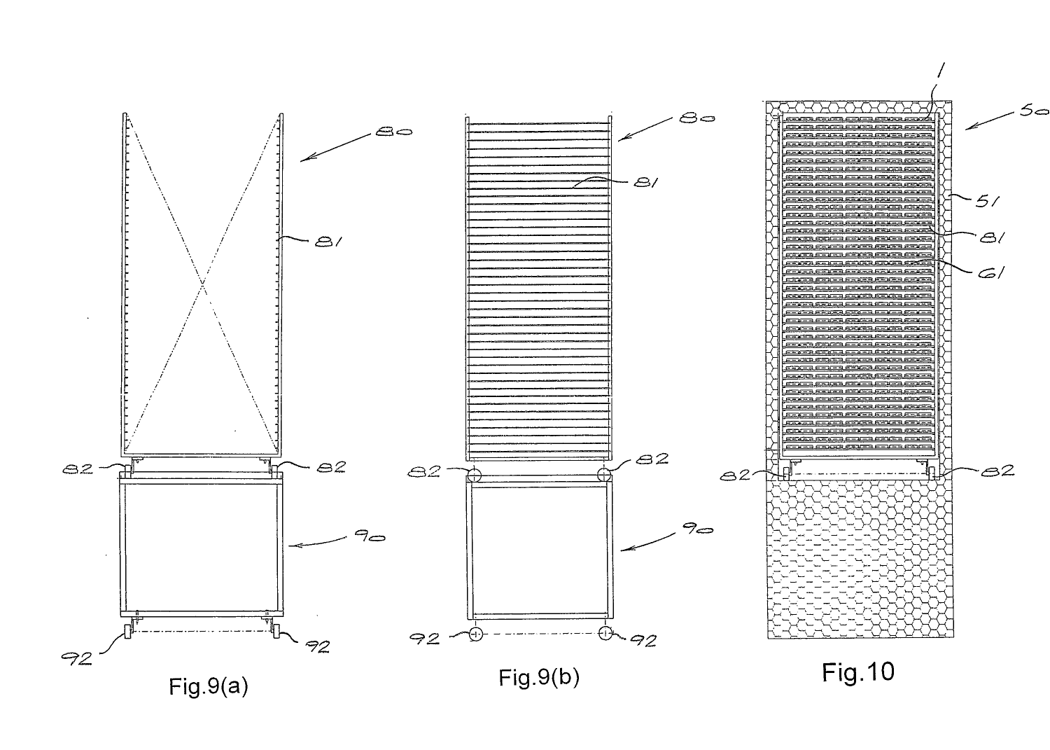

The preferred process of manufacturing the product is characterized by setting the pads from liquid condition in the packaging units, which has significant time and cost advantages. The process includes to following steps and is described with reference to FIGS. 5 to 10:
1. 1 kg PVA and 11 kg water is weighed into a heating vessel.
2. The vessel mixes the ingredients well with two stirrers and heats the ingredients at 80 degrees Celsius for 12 hours until the PVA has dissolved.
3. The vessel cools the solution down to room temperature.
4. 0.12 kg Fitobroside, 0.24 kg Regu-Age, 0.09 kg Collasol, 0.06 kg panthenol, 0.24kg Aloe Ferox powder and 0.06 kg Kemaben is added to the mixture and further mixed.

5. 6 ml of the cooled solution with added ingredients is injected into the packaging units 1 (6 ml per slice or 6 ml per side) which have been positioned onto the trays 60.
6. The packaging units 1 get closed and the trays 60 get inserted into a specially designed rack 80. The rack 80 is placed into a refrigeration unit (freezer) 50 (−15 degrees Celsius) for two hours. The rack 80 is taken out of the freezer 50 and the product allowed to thaw at room temperature (20 to 24 degrees Celcius) for an hour. The process gets repeated three times (freeze, thaw).
7. The product is ready after the last thaw, the packaging units 1 clipped closed and ready for sealing.

The trays 80 that are used for holding the packaging units 1 have the following features:

A 630×710×2 mm Stainless Steel refrigeration tray 60 locates 28 injection moulded packaging units 1 per tray 60. These trays 60 are designed to hold 28 packaging units 1 firmly in place (flat and open) by means of bars 71 which engage with the packaging units 1, so that the liquid hydrogel polymer with ingredients solution can be injected into the containers (6 ml per slice) in liquid form before the freeze/thaw part of the process. Due to the fact that the packaging forms part of the manufacturing process and the packaging units mould the treatment pads, the packaging units 1 have to be horizontally flat when the liquid gets injected into them.

A trolley 80 (mild steel fabrication and painted) is designed to hold 25 trays 60 by means of ribs 81 adapted to receive the trays 60. The trays 60, once loaded with packaging units 1 and fill, get inserted onto the trolley 80. This trolley 80 is then placed into the fridge 50 for freezing. The trolley 80 is adapted to fit on top of an assembly 90 allowing for insertion of the trolley 80 in the fridge 50. Both the trolley 80 and the assembly 90 are fitted with castor wheels 82 and 92 to facilitate this operation. The trolley 80 is removed out of the fridge 50 for thawing and the process repeated 3 times.

This trolley 80 is designed to perfectly fit the load space 52 of standard industrial fridge 50 and more trolleys 80 and trays 60 will be used as the demand increases. One trolley 80 fits one fridge 50 and therefore makes 1 batch of product (700 pairs of treatment pads).

The pad may be specialised to differentiate the product: e.g. Orange packaging and pale orange pads with vitamin C for dark circles; yellow packaging and pale yellow pads with vitamin A for anti-ageing; blue packaging and pale blue pads with Aloesin for puffiness etc.

The pad that is produced is soft, translucent, moist, rubbery and gel-like, preferably oval in shape that covers the entire eye region. The pads can be used in an inclined or upright position and do not require the user to be recumbent. The pad may be removed from the container for use and can be returned to the container for further use later.

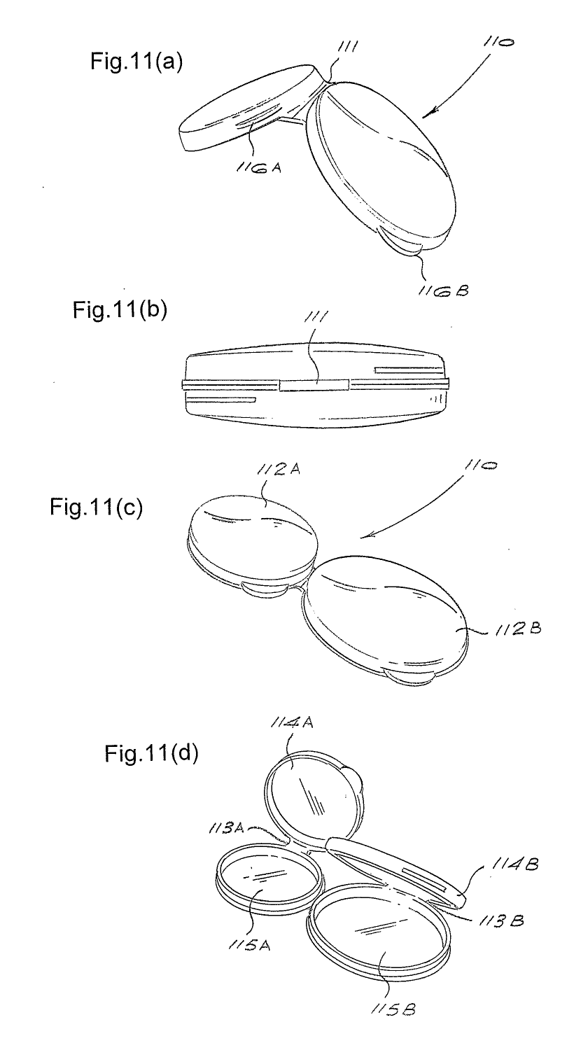

In FIGS. 11 (a-d) the packaging unit 110 comprises a hinge 111 conjoining two containers 112A and 112B. the containers comprise a lid 114A and 114B joined by a hinge 113A and 113B to a base 115A and 115B. The lids 114A and 114B frictionally engage with the bases 115A and 115B to seal to a closed position. They may be opened by way of a clip 116A and 116B.

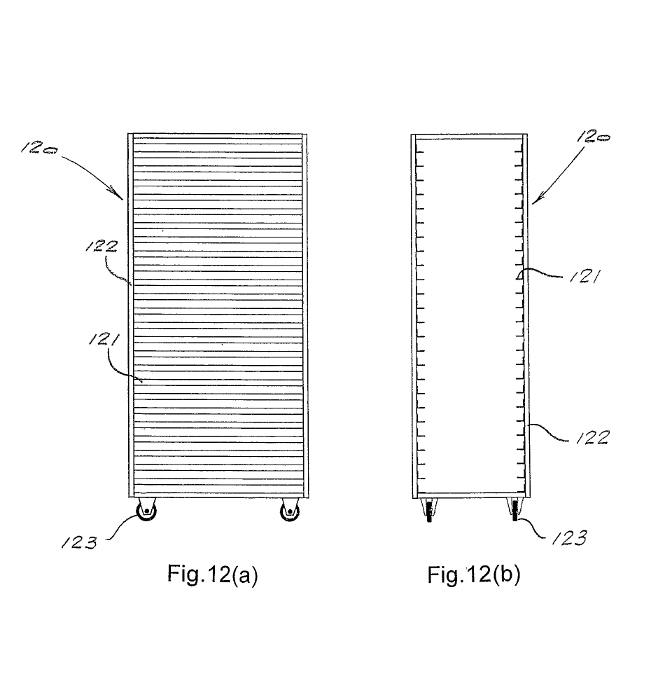

In FIGS. 12(a) and 12(b) a trolley assembly 120 comprises a tower 122 of trays racks 121 mounted on wheels 123. The tray racks 121 are adapted to received trays (not shown) upon which packaging units and/or formed, filled and sealed packages may be placed ready for the freeze thaw cycle.

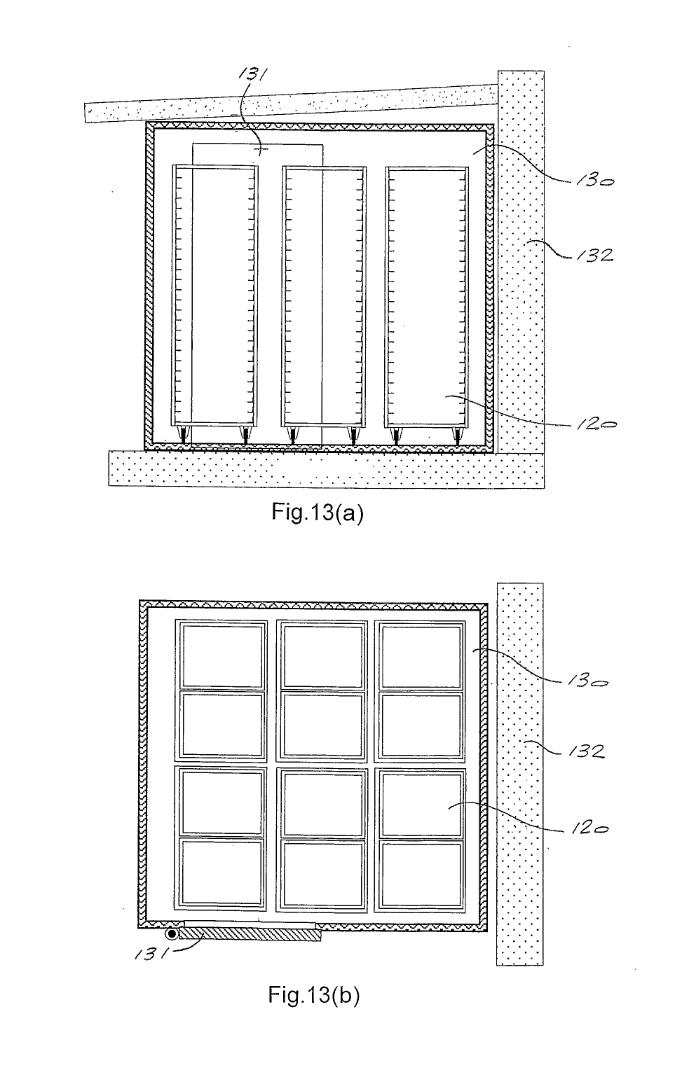

In FIGS. 13(a) and 13(b) a walk-in freezer 130 is shown which is located within a structure 132. The freezer includes a door 131. Several trolley assemblies 120 are located within the freezer 130.

1. A treatment pad comprising:
0.1 to 90% weight (% wt) of a hydrophilic polymer selected from the group consisting of polyvinyl alcohol (PVA), polyethylene glycol (EG), polyacrylamide (PAAm), polyacrylic acid (PAAc), polyhydroxyethyl methacrylate (PHEMA) and polyethylene oxide (PEO);
0.01 to 0.99% wt preservative; and
the balance being water.

2. A treatment pad according to claim 1 wherein the treatment pad is adapted to treat the eye and/or regions of tissue surrounding the eye.

3. A treatment pad according to claim 1 wherein the pad is self supporting.

4. A treatment pad according to claim 1 wherein the pad is ergonomically shaped to be accommodated within an eye well of a user thereof.

5. A treatment pad according to claim 1 wherein the pad is adapted to adhere to skin for at least 30 seconds in the vertical or substantially vertical position.

6. A treatment pad according to claim 1 wherein the hydrophilic polymer is present in an amount greater than 1% wt.

7. A treatment pad according to any previous claim 1 wherein the hydrophilic polymer is present in an amount less than 60% wt.

8. A treatment pad according to claim 1 wherein the hydrophilic polymer is polyvinyl alcohol (PVA).

9. A treatment pad according to claim 1 wherein the preservative is selected from those effective against gram negative and/or gram positive bacteria, yeasts and/or moulds or other unwanted life forms which may occur in or on the pad.

10. A treatment pad according to claim 1 wherein the preservative is selected from the group consisting of parabenzoates including methyl, ethyl, butyl and propyl parabenzoates, glycols, including propylene glycol and ureas including diazolidinyl urea.

11. A treatment pad according to claim 1 wherein the pad includes further ingredients for ameliorating disorders or unsightly marking of tissue and/or skin.

12. A treatment pad according to claim 11 wherein the further ingredients are selected from derivatives from the Aloe plant including Aloe Vera, Aloe Arborescence and Aloe Ferox, provitamins including panthenol, lipid dispersions, proteins, quinine and cinchona derivatives, suitable emollients, a source of collagen, vitamins, including vitamins A, B, C, D and E, green tea, cucumber extract, bulbine extract, stabilized peroxides and radical waters.

13. A treatment pad according to claim 1 wherein the pad contains suitable dyes, perfumes and/or other adjuvants.

14. A treatment pad according to claim 1 wherein the further ingredients, dyes, perfumes and adjuvants are present in an amount of from 0.01 to 20% wt each.

15. A treatment pad according to claim 1 wherein the pad includes plant material and/or extracts from locally grown indigenous plants in South Africa including fynbos and rooibos varieties including Buchu (Agathosma Betulina), Cape May (Coleonema Album), Zinziba (Lippia Javanica), Cape Chamomile (Eriocephalus Punctulatus), Cape geranium (Pelargoneum Graveolens) and Cape Snowbush (Eriocephalus Africanus).

16. A product comprising a treatment pad according to claim 1 and a packaging unit which comprises at least one container with a lid.

17. A product according to claim 16 wherein the packaging unit is adapted to receive at least two treatment pads.

18. A product according to claim 16 wherein the lid of the packaging unit engages with the container to substantially seal the contents of the container from the ambient, when the lid is closed.

19. A product according to claim 1 wherein the packaging unit comprises two conjoined containers, each with a base and a hinged lid, the containers being conjoined by a hinge that allows the containers to be hinged from an open position with respect to each other with their lids facing upwards to a position in which they are contiguous with each other, back to back.

20. A product according to claim 1 wherein the treatment pad may either be directly received in the container or the container may receive a package containing the treatment pad.

21. A method of preparation of a treatment pad according to claim 1 which comprises the steps of mixing the hydrophilic polymer, preservative and water and subjecting the mixture to at least one freeze thaw cycle.

22. A method according to claim 21 wherein the water and hydrophilic polymer are added in a heating vessel and heated to between 60 and 100 degrees Celcius (deg C.) for between 1 and 14 hours.

23. A method according to claim 22 wherein after cooling to approximately 40 deg C., the remaining ingredients are added and mixed to create a mixture.

24. A method according to 23 wherein a suitable aliquot of the mixture is added to a container.

25. A method according to claim 24 wherein the containers containing the mixture are subject to a predetermined number of freeze thaw cycles.

26. An apparatus comprising a tray adapted to receive a plurality of packaging units.

27. An apparatus as claimed in claim 26 wherein the apparatus is dimensioned to be accommodated within a refrigerator suitable for use in a freeze thaw processes.

28. (canceled)

29. (canceled)

30. (canceled)

31. (canceled)
